(19) United States
(12) Patent Application Publication
(10) Pub. No.: US 2008/0032307 A1
(43) Pub. Date: Feb. 7, 2008

(54) Use of Single-Stranded Nucleic Acid Binding Proteins In Sequencing

(75) Inventor: Philip R. Buzby, Brockton, MA (US)

Correspondence Address: COOLEY GODWARD KRONISH LLP;ATTN: Patent Group, Suite 1100, 777 - 6th Street, NW, WASHINGTON, DC, 20001, US

(73) Assignee: HELICOS BIOSCIENCES, INC., Cambridge, MA (US)

(21) Appl. No.: 11/843,712

(22) Filed: Aug. 23, 2007

Related U.S. Application Data
(63) Continuation of application No. 10/900,711, filed on Jul. 28, 2004, now abandoned.

(51) Int. Cl. C12Q 1/68

(52) U.S. Cl. 435/6

(57) ABSTRACT

The invention provides methods for stabilizing a nucleic acid sequencing reaction. Generally, methods of the invention include exposing a target nucleic acid to a single-stranded nucleic acid binding protein and performing a sequencing reaction.

### TECHNICAL FIELD OF THE INVENTION

The present invention relates to methods for stabilizing a nucleic acid sequencing reaction. More specifically, the present invention relates to methods for sequencing a target nucleic acid comprising exposing a target nucleic acid to a single-stranded nucleic acid binding protein.

### BACKGROUND OF THE INVENTION

Completion of the human genome has paved the way for important insights into biologic structure and function. Knowledge of the human genome has given rise to inquiry into individual differences, as well as differences within an individual, as the basis for differences in biological function and dysfunction. For example, single nucleotide differences between individuals, called single nucleotide polymorphisms (SNPs), are responsible for dramatic phenotypic differences. Those differences can be outward expressions of phenotype or can involve the likelihood that an individual will get a specific disease or how that individual will respond to treatment. Moreover, subtle genomic changes have been shown to be responsible for the manifestation of genetic diseases, such as cancer. A true understanding of the complexities in either normal or abnormal function will require large amounts of specific sequence information.

An understanding of cancer also requires an understanding of genomic sequence complexity. Cancer is a disease that is rooted in heterogeneous genomic instability. Most cancers develop from a series of genomic changes, some subtle and some significant, that occur in a small subpopulation of cells. Knowledge of the sequence variations that lead to cancer will lead to an understanding of the etiology of the disease, as well as ways to treat and prevent it. An essential first step in understanding genomic complexity is the ability to perform high-resolution sequencing.

Various approaches to nucleic acid sequencing exist. One conventional way to do bulk sequencing is by chain termination and gel separation, essentially as described by Sanger et al., Proc. Natl. Acad. Sci., 74(12): 5463-67 (1977). That method relies on the generation of a mixed population of nucleic acid fragments representing terminations at each base in a sequence. The fragments are then run on an electrophoretic gel and the sequence is revealed by the order of fragments in the gel. Another conventional bulk sequencing method relies on chemical degradation of nucleic acid fragments. See, Maxam et al., Proc. Natl. Acad. Sci., 74: 560-564 (1977). Finally, methods have been developed based upon sequencing by hybridization. See, e.g., Drmanac, et al., Nature Biotech., 16: 54-58 (1998).

There have been many proposals to develop new sequencing technologies based on single-molecule measurements, generally either by observing the interaction of particular proteins with DNA or by using ultra high resolution scanned probe microscopy. See, e.g., Rigler, et al., DNA-Sequencing at the Single Molecule Level, Journal of Biotechnology, 86(3): 161 (2001); Goodwin, P. M., et al., Application of Single Molecule Detection to DNA Sequencing. Nucleosides & Nucleotides, 16(5-6): 543-550 (1997); Howorka, S., et al., Sequence-Specific Detection of Individual DNA Strands using Engineered Nanopores, Nature Biotechnology, 19(7): 636-639 (2001); Meller, A., et al., Rapid Nanopore Discrimination Between Single Polynucleotide Molecules, Proceedings of the National Academy of Sciences of the United States of America, 97(3): 1079-1084 (2000); Driscoll, R. J., et al., Atomic-Scale Imaging of DNA Using Scanning Tunneling Microscopy. Nature, 346(6281): 294-296 (1990). Although none of these proposed methods have been demonstrated experimentally, they are interesting because they promise high sensitivity at reduced cost, and in some cases, a high degree of parallelization as well. Unlike conventional sequencing technologies, their speed and read-length would not be inherently limited by the resolving power of electrophoretic separation.

Other methods proposed for single molecule sequencing comprise detecting individual nucleotides incorporated during a template-dependant synthesis reaction (i.e., so-called, “sequencing by synthesis”). As applied to single molecule sequencing, current sequencing-by-synthesis methods fail to consistently provide a detectable and accurate signal indicative of the incorporation of a single nucleotide into a single template/primer complex. Indeed, the application of sequencing-by-synthesis techniques to single molecule sequencing has proven difficult in that the optimal conditions or measured enzyme kinetics for a sequencing reaction performed in bulk solution are unlikely to be the same for single molecules. For example, minor steric complications caused by modified nucleotide bases or base analogs, such as large fluorophore labeled nucleotide bases, in bulk sequencing frequently pose insurmountable obstacles in single molecule sequencing. Such steric complications may be caused by, for example, the difficulty in incorporating modified nucleotide bases or base analogs into the tight and compact formation of nucleic acid chains in their natural state.

Furthermore, the extraordinarily high linear data density of DNA (3.4 Å/base) has been a major obstacle in the development of a single-molecule DNA sequencing technology. Scanned probe microscopes have not yet been able to demonstrate simultaneously the resolution and chemical specificity needed to resolve individual bases. Other proposals turn to nature for inspiration and seek to combine optical techniques with enzymes that have been fine-tuned by evolution to operate as machines that assemble and disassemble DNA with single-base resolution.

As discussed earlier, conventional nucleotide sequencing is accomplished through bulk techniques. Bulk sequencing techniques are not useful for the identification of subtle or rare nucleotide changes due to the many cloning, amplification and electrophoresis steps that complicate the process of gaining useful information regarding individual nucleotides. As such, research has evolved toward methods for rapid sequencing, such as single molecule sequencing technologies. The ability to sequence and gain information from single molecules obtained from an individual patient is the next milestone for genomic sequencing. However, effective diagnosis and management of important diseases through single molecule sequencing is impeded by lack of cost-effective tools and methods for screening individual molecules.

A need therefore exists for more effective and efficient methods for single molecule nucleic acid sequencing.

### SUMMARY OF THE INVENTION

The invention provides methods for stabilizing or facilitating a nucleic acid sequencing reaction, or analysis of such a reaction. In general terms, the invention provides methods for sequencing a nucleic acid comprising exposing a target nucleic acid template to a single-stranded nucleic acid binding protein and performing template-dependent nucleic acid synthesis.

In one embodiment, the invention provides a method for stabilizing a nucleic acid sequencing reaction by exposing a reaction mixture comprising a target nucleic acid template, a polymerase and a primer to a single-stranded nucleic acid binding protein. Stabilizing the reaction results in improved speed, accuracy, and precision of the reaction. For example, upon stabilization of the reaction, the polymerase may exhibit improved speed, fidelity or processivity. A single-stranded nucleic acid binding protein stabilizes the reaction by, for example, keeping the single-stranded nucleic acid in a linear conformation and preventing the coiling or formation of tertiary structures that inhibit polymerase-catalyzed extension of the primer. Any polymerase that catalyzes the incorporation of a nucleotide into a primer in a template-dependent fashion is useful in methods of the invention. In one embodiment, a polymerase having either a decreased 5′ to 3′ or a decreased 3′ to 5′ proofreading ability is used.

According to one embodiment, the invention provides methods for sequencing a polynucleotide comprising stabilizing a nucleic acid template/primer complex with a single-stranded nucleic acid binding protein, exposing the complex to a polymerase and at least one nucleotide capable of extending the primer, and determining whether the nucleotide has extended the primer. The steps are repeated in order to compile a sequence of the polynucleotide based upon the order of nucleotides added to the primer. In a preferred embodiment, unincorporated nucleotide is removed prior to repeating the exposing and determination steps.

Nucleotides useful in the invention include any nucleotide or nucleotide analog, whether naturally-occurring or synthetic. For example, preferred nucleotides are adenine, cytosine, guanine, uracil, or thymine bases; xanthine or hypoxanthine, 5-bromouracil, 2-aminopurine, deoxyinosine, or methylated cytosine, such as 5-methylcytosine, and N4-methoxydeoxycytosine. Also included are bases of polynucleotide mimetics, such as methylated nucleic acids, e.g., 2′-O-methRNA, peptide nucleic acids, modified peptide nucleic acids, and any other structural moiety that can act substantially like a nucleotide or base, for example, by exhibiting base-complementarity with one or more bases that occur in DNA or RNA and/or being capable of base-complementary incorporation, and includes chain-terminating analogs.

Nucleotides particularly useful in the invention comprise detectable labels. Labeled nucleotides include any nucleotide that has been modified to include a label that is directly or indirectly detectable. Preferred labels include optically-detectable labels, including fluorescent labels or fluorophores, such as fluorescein, rhodamine, derivatized rhodamine dyes, such as TAMRA, phosphor, polymethadine dye, fluorescent phosphoramidite, texas red, green fluorescent protein, acridine, cyanine, cyanine 5 dye, cyanine 3 dye, 5-(2′-aminoethyl)-aminonaphthalene-1-sulfonic acid (EDANS), BODIPY, 120 ALEXA, or a derivative or modification of any of the foregoing.

In one embodiment, the nucleotide is labeled with a first label and the single-stranded nucleic acid binding protein or the polymerase is labeled with a second label. In another embodiment, a single-stranded nucleic acid binding protein is fluorescently labeled to facilitate the detection of labeled nucleotides as they are incorporated into the primer. In some embodiments, the invention utilizes fluorescence resonance energy transfer (FRET) as a detection scheme for determining the base type incorporated into the growing primer. Fluorescence resonance energy transfer in the context of sequencing is described generally in Braslavasky, et al., Proc. Nat'l Acad. Sci., 100: 3960-3964 (2003), incorporated by reference herein. Essentially, in one embodiment, a donor fluorophore is attached to either the primer, polymerase, or a single-stranded nucleic acid binding protein. Nucleotides added for incorporation into the primer comprise an acceptor fluorophore that can be activated by the donor when the two are in proximity. Activation of the acceptor causes it to emit a characteristic wavelength of light and also quenches the donor. In this way, incorporation of a nucleotide in the primer sequence is detected by detection of acceptor emission. Of course, nucleotides labeled with a donor fluorophore also are useful in methods of the invention; FRET-based methods of the invention only require that a donor and acceptor fluorophore pair are used, a labeled nucleotide comprising one fluorophore and either the single-stranded nucleic acid binding protein or the polymerase comprising the other. Such labeling techniques may result in a coincident fluorescent emission of the labels of the nucleotide and the single-stranded nucleic acid binding protein or polymerase, or the fluorescent emission of only one of the labels.

In one embodiment of the invention, whether the nucleotide has been incorporated into the primer is determined by detecting the presence or absence of the label on a labeled nucleotide. Such detection may be made directly, indirectly, optically or otherwise. In a preferred embodiment, after detection, the label is rendered undetectable by removing the label from the nucleotide or extended primer, neutralizing the label, or masking the label. In certain embodiments, methods according to the invention provide for neutralizing a label by photobleaching. This is accomplished by focusing a laser with a short laser pulse, for example, for a short duration of time with increasing laser intensity. In other embodiments, a label is photocleaved. For example, a light-sensitive label bound to a nucleotide is photocleaved by focusing a particular wavelength of light on the label. Generally, it may be preferable to use lasers having differing wavelengths for exciting and photocleaving. Labels also can be chemically cleaved. Labels may be removed from a substrate using reagents, such as NaOH or other appropriate buffer reagent.

In a preferred embodiment of the invention, a target nucleic acid template is attached to a substrate such that individual nucleic acids are optically resolvable. Each member of the plurality is attached to a surface, such as glass or fused silica, preferably by covalent attachment. One skilled in the art will understand that target nucleic acids can be attached to any surface that allows primer extension, and preferably, to any surface suitable for detecting incorporation of nucleotides or nucleotide analogs. As such, in some embodiments, each member of the plurality of target nucleic acids is covalently attached to a surface that has reduced background fluorescence with respect to glass, polished glass or fused silica. Examples of surfaces appropriate for the invention include polytetrafluoroethylene or a derivative of polytetrafluoroethylene, such as silanized polytetrafluoroethylene. In addition, in preferred embodiments of the invention, target nucleic acids are spaced apart on a substrate such that each target is optically resolvable. In practice, for example, the target may be optically resolved by detecting a fluorescent label attached to the nucleotide.

In a preferred embodiment, a single-stranded nucleic acid binding protein is attached to a substrate. In this embodiment, a nucleic acid template and a polymerase are exposed to a labeled nucleotide in the presence of the substrate bound single-stranded nucleic acid binding protein. The sequencing reaction is carried out with the nucleic acid template attached to the single-stranded nucleic acid binding protein which itself is attached to a surface, thus anchoring the nucleic acid template without the need for additional reagents such as streptavidin. In addition, anchoring the nucleic acid template with a single-stranded nucleic acid binding protein can be accomplished without modifying the template to comprise biotin.

A detailed description of embodiments of the invention is provided below. Other embodiments of the invention are apparent upon review of the detailed description that follows.

### BRIEF DESCRIPTION OF THE DRAWINGS

FIG. 1 shows a representative sequencing-by-synthesis reaction of a single-stranded nucleic acid facilitated by a single-stranded nucleic acid binding protein.

FIG. 2 shows an optical detection system utilizing an intensified charge couple device camera for detecting the incorporation of labeled nucleotides to a primer.

FIG. 3 depicts an exemplary single molecule sequencing reaction conducted in the presence of a single-stranded nucleic acid binding protein attached to a substrate. The exemplary reaction is conducted so that any incorporation events are individually optically resolvable by detecting labeled nucleotides incorporated into a primer on the substrate.

FIG. 4 depicts an exemplary stepwise primer extension reaction for sequencing a target nucleic acid template by exposing the template to a single-stranded nucleic acid binding protein, labeled nucleotides and a primer.

### DETAILED DESCRIPTION OF THE INVENTION

Single molecule sequencing benefits from highly-sensitive and cost-effective tools and methods to provide rapid and accurate results. Single molecule sequencing provides sequence-specific genomic information that is relevant to both normal and diseased function. As such, the fidelity of incorporation of the nucleotides to a primer is important for reliably analyzing subtle genomic events. The methods and tools discussed herein provide optimal conditions and kinetics for conducting single molecule sequencing reactions.

One of the difficulties with obtaining accurate and reproducible data from single molecule sequencing reactions is detecting incorporation events from primer extension reactions. For single molecule sequencing, there are a number of factors that interfere with incorporation of nucleotides to a primer. For example, the fidelity of nucleotide incorporation depends on conditions such as temperature and the complexity of template that is to be interrogated.

In cells, a single-stranded nucleic acid binding protein binds to the lagging single-stranded nucleic acid created by a DnaB helicase. A single-stranded nucleic acid binding protein prevents the target nucleic acid (such as DNA) from forming secondary structures thereby stabilizing the target nucleic acid to facilitate the rate of synthesis rate. Furthermore, by limiting the target nucleic acid from forming secondary structures, a single-stranded nucleic acid binding protein enhances the ability of a polymerase to correct any errors during synthesis.

Single-stranded nucleic acid binding proteins are representative of a class of proteins that has a high affinity for, or preferentially binds to, single-stranded nucleic acids and interferes with the formation of secondary structures with the single-stranded nucleic acids. The preferred binding of single-stranded binding proteins to single-stranded nucleic acids occurs irrespective of the nucleic acid sequence. A single-stranded nucleic acid binding protein binds a single-stranded nucleic acid stoichiometrically in an amount that depends on the particular single-stranded nucleic acid binding protein. A single-stranded nucleic acid binding protein also reduces the melting temperature of double-stranded nucleic acid and increases the processivity of a polymerase during primer extension.

Various single-stranded nucleic acid binding proteins are known in the art, and include members such as the E. coli single-stranded nucleic acid binding protein, T4 gene 32 protein (T4 gp32), T4 gene 44/62 protein, T7 SSB, coliphage N4 SSB, adenovirus DNA binding protein, calf thymus unwinding protein, and purified single-stranded nucleic acid binding protein from T. thermophilus strain HB8. See Celia et al., Nuc. Acid. Res., 31 (22), 6473-6480. A single-stranded nucleic acid binding protein may come from any source, either eukaryotic or prokaryotic, and may include a single-stranded DNA binding protein, a single-stranded RNA binding protein, a topoisomerase, and double-stranded (e.g., DNA) unwinding proteins. Single-stranded nucleic acid binding proteins that are derived by isolation of mutants or by manipulation of cloned single-stranded nucleic acid binding protein-encoding genes are also contemplated by methods and tools according to the invention. A single-stranded nucleic acid binding protein can be used alone or in combination with other single-stranded nucleic acid binding proteins to stabilize or facilitate a nucleic acid sequencing reaction.

The amount of one or more single-stranded nucleic acid binding proteins for use in the disclosed methods depends on the amount of nucleic acid (single or double stranded) present in the mixture, as single-stranded nucleic acid binding protein binds to nucleic acids stoichiometrically. For example, Eco single-stranded nucleic acid binding protein binds single-stranded nucleic acid to a maximum of about one single-stranded nucleic acid binding protein site per 33 to 65 base nucleotides. Salt concentration also influences the binding properties of single-stranded nucleic acid binding protein. Typically, an amount of about 1 ng to about 10 ug of single-stranded nucleic acid binding protein per 100 ng of target nucleic acid effectively binds target nucleic acids, although ranges below and above also may be effective depending on factors such as the species of single-stranded nucleic acid binding protein, salt concentration of the reaction, desired speed of reaction, or amount of polymerase introduced, for example.

A single-stranded nucleic acid binding protein can also be bound, covalently or otherwise, to a label. For example, a single-stranded nucleic acid binding protein can comprise a detectable label. The ability to resolve and detect nucleotide incorporation into a primer is of the utmost importance when performing single molecule sequencing reactions. As such, methods of the invention include a detectable labeling method that does not impact the fidelity of the overall nucleic acid sequencing reaction and that does not provide excessive background noise or illumination that interferes with the detection of incorporated labeled nucleotides. One detectable labeling method includes FRET or the use of donor and acceptor fluorophores. In addition to or instead of labeling donor and acceptors fluorophores on nucleotides, according to the invention, a single-stranded nucleic acid binding protein can be labeled with a fluorophore to create a detectable event. The detectable event results from an interaction between a labeled nucleotide incorporated into the primer and the fluorophore of the single-stranded nucleic acid binding protein when they are proximately located, whereby a photon is either released or captured.

Methods according to the invention provide for more efficient and error-free sequencing with greater applications in disease detection and diagnosis for individual analysis. A target nucleic acid for analysis may be obtained directly from a patient, and such methods are particularly useful in connection with a variety of biological samples, such as blood, urine, cerebrospinal fluid, seminal fluid, saliva, breast nipple aspirate, sputum, stool and biopsy tissue. Especially preferred are samples of luminal fluid because such samples are generally free of intact, healthy cells. However, any tissue or body fluid specimen may be used according to methods of the invention.

A target nucleic acid can come from a variety of sources. For example, nucleic acids can be naturally occurring DNA or RNA isolated from any source, recombinant molecules, cDNA, or synthetic analogs, as known in the art. For example, the target nucleic acid may be genomic DNA, genes, gene fragments, exons, introns, regulatory elements (such as promoters, enhancers, initiation and termination regions, expression regulatory factors, expression controls, and other control regions), DNA comprising one or more single-nucleotide polymorphisms (SNPs), allelic variants, and other mutations. Also included is the full genome of one or more cells, for example cells from different stages of diseases such as cancer. The target nucleic acid may also be mRNA, tRNA, rRNA, ribozymes, splice variants, antisense RNA, and RNAi. Also contemplated according to the invention are RNA with a recognition site for binding a polymerase, transcripts of a single cell, organelle or microorganism, and all or portions of RNA complements of one or more cells, for example, cells from different stages of development or differentiation, and cells from different species. Nucleic acids can be obtained from any cell of a person, animal, plant, bacteria, or virus, including pathogenic microbes or other cellular organisms. Individual nucleic acids can be isolated for analysis.

Methods according to the invention provide for the determination of the sequence of a single molecule, such as a single-stranded target nucleic acid, utilizing single-stranded nucleic acid binding protein at various points in the procedure. Generally, target nucleic acids can have a length of about 5 bases, about 10 bases, about 20 bases, about 30 bases, about 40 bases, about 50 bases, about 60 bases, about 70 bases, about 80 bases, about 90 bases, about 100 bases, about 200 bases, about 500 bases, about 1 kb, about 3 kb, about 10 kb, or about 20 kb and so on. Preferred methods of the invention provide for a sequencing and detection system directed towards non-amplified and/or non-purified target nucleic acid sequences.

Methods according to the invention include exposing a target nucleic acid to a primer in the presence of a single-stranded nucleic acid binding protein. The primer may be selected to bind to complementary regions of the template or may be fixed onto an end of the template itself. In general, the primer is complementary to at least a portion of the target nucleic acid. The target nucleic acid also is exposed to a polymerase, at least one nucleotide or nucleotide analog allowing for extension of the primer, and a single-stranded nucleic acid binding protein. A nucleotide or nucleotide analog includes any base or base-type including adenine, cytosine, guanine, uracil, or thymine bases. In addition, additional nucleotide analogs include xanthine or hypoxanthine, 5-bromouracil, 2-aminopurine, deoxyinosine, or methylated cytosine, such as 5-methylcytosine, N4-methoxydeoxycytosine, and the like. Also included are bases of polynucleotide mimetics, such as methylated nucleic acids, e.g., 2′-O-methRNA, peptide nucleic acids, modified peptide nucleic acids, and any other structural moiety that can act substantially like a nucleotide or base, for example, by exhibiting base-complementarity with one or more bases that occur in DNA or RNA and/or being capable of base-complementary incorporation.

Methods of the invention also include detecting incorporation of the nucleotide or nucleotide analog in the primer and, repeating the exposing, conducting and/or detecting steps to determine a sequence of the target nucleic acid. A researcher can compile the sequence of a complement of the target nucleic acid based upon sequential incorporation of the nucleotides into the primer. Similarly, the researcher can compile the sequence of the target nucleic acid based upon the complement sequence.

Also, a nucleotide analog can be modified to remove, cap or modify the 3′ hydroxyl group. As such, in certain embodiments, methods of the invention can include, for example, the step of removing the 3′ hydroxyl group from the incorporated nucleotide or nucleotide analog. By removing the 3′ hydroxyl group from the incorporated nucleotide in the primer, further extension is halted or impeded. In certain embodiments, the modified nucleotide can be engineered so that the 3′ hydroxyl group can be removed and/or added by chemical methods.

In addition, a nucleotide analog can be modified to include a moiety that is sufficiently large to prevent or sterically hinder further chain elongation by interfering with the polymerase, thereby halting incorporation of additional nucleotides or nucleotide analogs. Subsequent removal of the moiety, or at least the steric-hindering portion of the moiety, can concomitantly reverse chain termination and allow chain elongation to proceed. In some embodiments, the moiety also can be a label. As such, in those embodiments, chemically cleaving or photocleaving the blocking moiety may also chemically-bleach or photo-bleach the label, respectively.

The methods according to the invention can provide de novo sequencing, sequence analysis, DNA fingerprinting, polymorphism identification, for example single nucleotide polymorphisms (SNP) detection, as well as applications for genetic cancer research. Applied to RNA sequences, methods according to the invention also can identify alternate splice sites, enumerate copy number, measure gene expression, identify unknown RNA molecules present in cells at low copy number, annotate genomes by determining which sequences are actually transcribed, determine phylogenic relationships, elucidate differentiation of cells, and facilitate tissue engineering. The methods according to the invention also can be used to analyze activities of other biomacromolecules such as RNA translation and protein assembly. Certain aspects of the invention lead to more sensitive detection of incorporated signals and faster sequencing.

A single-stranded nucleic acid binding protein can be used unbound to any other component, and/or it can be bound, covalently or adsorptively, to a substrate, surface, support or any array. In one embodiment, a target nucleic acid can be covalently attached to a substrate, surface, support or any array, such as glass or fused silica. For example, each member of the plurality of target nucleic acids can be covalently attached to a surface that has reduced background fluorescence with respect to glass, polished glass, fused silica or plastic. Examples of surfaces appropriate for the invention include, for example, polytetrafluoroethylene or a derivative of polytetrafluoroethylene, such as silanized polytetrafluoroethylene.

In another embodiment, a target nucleic acid also can be exposed to a single-stranded nucleic acid binding protein that is attached to a substrate, support, surface or array. The single-stranded nucleic acid binding protein can be covalently attached to a substrate, such as a surface that has a reduced background fluorescence with respect to glass, polished glass, fused silica or plastic. Examples of surfaces appropriate for the substrate include, for example, polytetrafluoroethylene or a derivative of polytetrafluoroethylene, such as silanized polytetrafluoroethylene. In this way, single-stranded nucleic acid binding proteins anchored to a substrate would bind the template nucleic acid and form a substrate-single-stranded nucleic acid binding protein/template complex, whereas nucleic acid sequencing of the template would commence as discussed herein.

The substrate, support, surface or array can be coated with single-stranded nucleic acid binding proteins substantially in its entirety. However, single-stranded nucleic acid binding proteins can be positioned on a substrate, support, surface or array in pre-determined positions, such that the nucleic acid templates attached to the binding proteins can be individually optically resolvable. Locations on a substrate, surface, support or array include a target nucleic acid that is linked thereto. In some embodiments, the locations include a primer, a target polynucleotide-primer complex, and/or a polymerase bound thereto. These moieties can be bound or immobilized on the surface of the substrate or array by covalent bonding, non-covalent bonding, ionic bonding, hydrogen bonding, van der Waals forces, hydrophobic bonding, or a combination thereof. The immobilizing may utilize one or more binding-pairs, including, but not limited to, an antigen-antibody binding pair, a streptavidin-biotin binding pair, photoactivated coupling molecules, and a pair of complementary nucleic acids. Furthermore, the substrate or support may include a semi-solid support (e.g., a gel or other matrix), and/or a porous support (e.g., a nylon membrane or other membrane). The surface of the substrate or support may be planar, curved, pointed, or any suitable two-dimensional or three-dimensional geometry.

A single molecule substrate or array describes a support or an array in which all or a subset of molecules of the array can be individually resolved and/or detected. According to invention, methods include the step of detecting incorporation of a nucleotide or nucleotide analog in a primer. Generally, the detection system includes any device that can detect and/or record light emitted from a nucleotide, from a single-stranded nucleic acid binding protein, from a target nucleic acid and/or a primer, and/or a polymerase. Accordingly, a detection system has single-molecule resolution or the ability to resolve one molecule from another. For example, in certain embodiments, the detection limit is in the order of a micron. Therefore, two molecules can be a few microns apart and be resolved, that is individually detected and/or detectably distinguished from each other.

Methods of the invention also include binding a single-stranded nucleic acid to a single-stranded nucleic acid binding protein on a substrate, such as a solid support. This allows for a sequencing reaction to occur without the addition of chemical reagents such as streptavidin that may interfere with an extension reaction or detection thereof. In this method, for example, a single-stranded nucleic acid binding protein is exposed to a solid substrate and a single-stranded nucleic acid (template) is introduced. Due to the high binding affinity of the single-stranded nucleic acid binding protein for the single-stranded nucleic acid template, the template securely attaches to the surface which comprises the single-stranded nucleic acid binding protein. As such, one advantage of the use of single-stranded nucleic acid binding proteins is that nucleic acid templates are not required to be modified to comprise a biotin or other binder to attach to a surface. The surface of the substrate may be coated with a single-stranded nucleic acid binding protein, or the single-stranded nucleic acid binding protein may be positioned on the surface. It is preferred that the single-stranded nucleic acid binding proteins are located such that the template is individually optically resolvable.

Certain embodiments of the invention are described in the following examples, which are not meant to be limiting.

### EXAMPLES

### Example 1

### Stabilizing a Nucleic Acid Sequencing Reaction

In this method, a target nucleic acid sequence (template) of a single-stranded nucleic acid is exposed and stabilized with a single-stranded nucleic acid binding protein. The template and single-stranded nucleic acid binding protein also are exposed to a primer, a polymerase, and nucleotides (or nucleotide analogs). First, a target nucleic acid is obtained from a patient using any of a variety of known procedures for extracting the nucleic acid. Although unnecessary for single molecule sequencing, the extracted nucleic acid can be optionally purified and then amplified to a concentration convenient for genotyping or sequencing work. Nucleic acid amplification methods are known in the art, such as polymerase chain reaction. Other amplification methods known in the art that can be used include ligase chain reaction, for example.

A single-stranded nucleic acid binding protein is selected to bind to the single stranded nucleic acid to stabilize the sequencing reaction. For example, a single-stranded nucleic acid binding protein may be purchased commercially, or purified from one of many identified sources, such as, T. thermophilus bacteria. A single-stranded nucleic acid binding protein also can be isolated from its source organism by standard biochemical methods involving cell lysis, protein chromatography, or other methods known in the art. The single-stranded nucleic acid binding protein can be selected to be substantially free of exonuclease activity. In addition, a single-stranded nucleic acid binding protein can be thermophilic or heat stable in high temperatures (e.g., greater than about 50-100 degrees Celsius). Furthermore, salt concentrations, including but not limited to divalent cation concentrations, may be manipulated to achieve optimal single-stranded nucleic acid binding protein stabilization of the single strand nucleic acid target.

Sequencing a target nucleic acid by synthesizing its complementary strand can include the step of hybridizing a primer to the target nucleic acid. Primer length can be selected to facilitate hybridization to a sufficiently complementary region of the template nucleic acid downstream of the region to be analyzed. The exact lengths of the primers depend on many factors, including temperature and source of primer.

If part of the region downstream of the sequence to be analyzed is known, a specific primer can be constructed and hybridized to this region of the target nucleic acid. Alternatively, if sequences of the downstream region on the target nucleic acid are not known, universal (e.g., uniform) or random primers may be used in random primer combinations. As another approach, a linker or adaptor can be joined to the ends of a target nucleic acid polynucleotide by a ligase and primers can be designed to bind to these adaptors. That is, a linker or adaptor can be ligated to at least one target nucleic acid of unknown sequence to allow for primer hybridization. Alternatively, known sequences may be biotinylated and ligated to the targets. In yet another approach, nucleic acid may be digested with a restriction endonuclease, and primers designed to hybridize with the known restriction sites that define the ends of the fragments produced.

Primers can be synthetically made using conventional nucleic acid synthesis techniques. For example, primers can be synthesized on an automated DNA synthesizer, e.g. an Applied Biosystems, Inc. (Foster City, Calif.) model 392 or 394 DNA/RNA Synthesizer, using standard chemistries, such as phosphoramidite chemistry, and the like. Alternative chemistries, e.g., resulting in non-natural backbone groups, such as phosphorothioate, phosphoramidate, and the like, may also be employed provided that, for example, the resulting oligonucleotides are compatible with the polymerizing agent. The primers can also be ordered commercially from a variety of companies which specialize in custom nucleic acids such as Operon, Inc. (Alameda, Calif.).

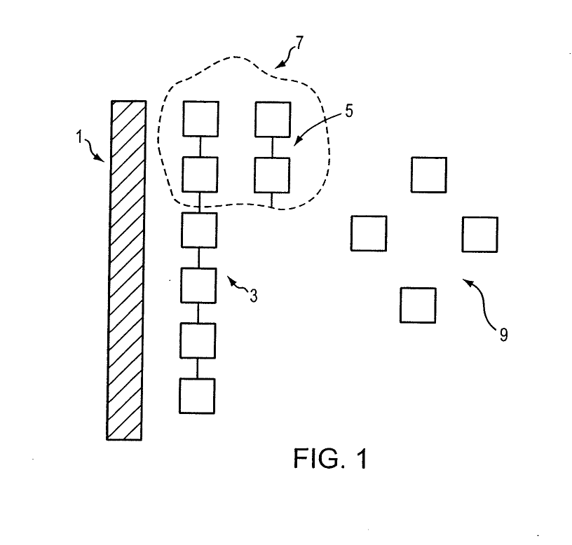

After preparing the target nucleic acid and optionally linking it on a substrate, primer extension reactions can be performed to analyze the target polynucleotide sequence by synthesizing its complementary strand. As shown in FIG. 1, a single-stranded nucleic acid binding protein 1 binds to a template 3 to stabilize the sequencing reaction. A concentration of single-stranded nucleic acid binding protein 1 is selected stoichiometrically such that a sufficient amount is added to bind all available templates. The single-stranded nucleic acid binding protein 1 selected stabilizes the single-stranded template 3 by inhibiting the formation of secondary conformations. A primer 5 that is selected to be substantially complementary to at least a portion of the template 3 is added along with a polymerase 7 to catalyze the binding of the primer 5 to the template 3 and the extension of the primer 5 in the presence of added nucleotides 9. Preferably, added nucleotides 9 are labeled so that incorporation events can be detected.

### Example 2

### Detecting Incorporation of a Nucleotide

A nucleic acid sequencing reaction is accomplished as in Example 1. In this instance, the primer includes a label. When hybridized to a nucleic acid molecule, the label facilitates locating the bound molecule through imaging. The primer can be labeled with a fluorescent labeling moiety (e.g., Cy3 or Cy5), or any other means used to label nucleotides. The detectable label used to label the primer can be different from the label used on the nucleotides or nucleotide analogs in the subsequent extension reactions. Suitable fluorescent labels include, but are not limited to, 4-acetamido-4′-isothiocyanatostilbene-2,2′disulfonic acid; acridine and derivatives: acridine, acridine isothiocyanate; 5-(2′-aminoethyl)aminonaphthalene-1-sulfonic acid (EDANS); 4-amino-N-[3-vinylsulfonyl)phenyl]naphthalimide-3,5 disulfonate; N-(4-anilino-1-naphthyl)maleimide; anthranilamide; BODIPY; Brilliant Yellow; coumarin and derivatives; coumarin, 7-amino-4-methylcoumarin (AMC, Coumarin 120), 7-amino-4-trifluoromethylcouluarin (Coumaran 151); cyanine dyes; cyanosine; 4′,6-diaminidino-2-phenylindole (DAPI); 5′5″-dibromopyrogallol-sulfonaphthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansylchloride); 4-dimethylaminophenylazophenyl-4′-isothiocyanate (DABITC); eosin and derivatives; eosin, cosin isothiocyanate, erythrosin and derivatives; erythrosin B, erythrosin, isothiocyanate; ethidium; fluorescein and derivatives; 5-carboxyfluorescein (FAM), 5-(4,6-dichlorotriazin-2-yl)aminofluorescein (DTAF), 2′,7′-dimethoxy-4′5′-dichloro-6-carboxyfluorescein (JOE), fluorescein, fluorescein isothiocyanate, QFITC, (XRITC); fluorescamine; IR144; IR1446; Malachite Green isothiocyanate; 4-methylumbelliferoneortho cresolphthalein; nitrotyrosine; pararosaniline; Phenol Red; B-phycoerythrin; o-phthaldialdehyde; pyrene and derivatives: pyrene, pyrene butyrate, succinimidyl 1-pyrene; butyrate quantum dots; Reactive Red 4 (Cibacron™ Brilliant Red 3B-A) rhodamine and derivatives: 6-carboxy-X-rhodamine (ROX), 6-carboxyrhodamine (R6G), lissamine rhodamine B sulfonyl chloride rhodamine (Rhod), rhodamine B, rhodamine 123, rhodamine X isothiocyanate, sulforhodamine B, sulforhodamine 101, sulfonyl chloride derivative of sulforhodamine 101 (Texas Red); N,N,N′,N′tetramethyl-6-carboxyrhodamine (TAMRA); tetramethyl rhodamine; tetramethyl rhodamine isothiocyanate (TRITC); riboflavin; rosolic acid; terbium chelate derivatives; Cy3; Cy5; Cy5.5; Cy7; IRD 700; IRD 800; La Jolta Blue; phthalo cyanine; and naphthalo cyanine.

The primer can be hybridized to the target nucleic acid before or after it is linked on a surface of a substrate or array. Primer annealing can be performed under conditions which are stringent enough to require sufficient sequence specificity, yet permissive enough to allow formation of stable hybrids at an acceptable rate. The temperature and time required for primer annealing depend upon several factors including base composition, length, and concentration of the primer; the nature of the solvent used, e.g., the concentration of DMSO, formamide, or glycerol; as well as the concentrations of counter ions, such as magnesium. Typically, hybridization with synthetic polynucleotides is carried out at a temperature that is approximately 5° C. to approximately 10° C. below the melting temperature (Tm) of the target polynucleotide-primer complex in the annealing solvent. However, according to methods of the invention, hybridization may be performed at much lower temperatures, such as for example 30-50° C. or 30-40° C. The annealing reaction can be complete within a few seconds.

Depending on the characteristics of the target template, a DNA polymerase, a RNA polymerase, or a reverse transcriptase can be used in the primer extension reactions. The incorporation of the labeled nucleotide or nucleotide analog then can be detected on the primer. A number of systems are available to detect this incorporation. Methods for visualizing single molecules of labeled nucleotides with an intercalating dye include, e.g., fluorescence microscopy. In some embodiments, the fluorescent spectrum and lifetime of a single molecule excited-state can be measured. Standard detectors such as a photomultiplier tube or avalanche photodiode can be used. Full field imaging with a two-stage image intensified charged couple device (CCD) camera can also used. Additionally, low noise cooled CCD can also be used to detect single fluorescent molecules.

The detection system for the signal may depend upon the labeling moiety used, which can be defined by the chemistry available. For optical signals, a combination of an optical fiber or CCD can be used in the detection step. In the embodiments where the substrate is itself transparent to the radiation used, it is possible to have an incident light beam pass through the substrate with the detector located opposite the substrate from the primer. For electromagnetic labels, various forms of spectroscopy systems can be used. Various physical orientations for the detection system are available and known in the art.

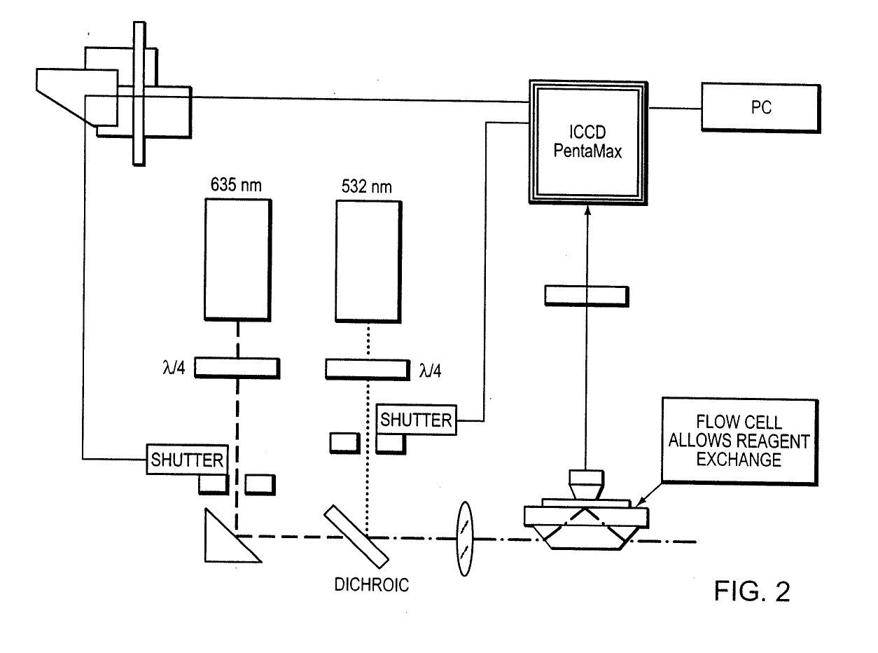

A number of approaches can be used to detect incorporation of fluorescently-labeled nucleotides into a single molecule. Optical systems include near-field scanning microscopy, far-field confocal microscopy, wide-field epi-illumination, light scattering, dark field microscopy, photoconversion, single and/or multiphoton excitation, spectral wavelength discrimination, fluorophore identification, evanescent wave illumination, and total internal reflection fluorescence (TIRF) microscopy. In general, methods involve detection of laser-activated fluorescence using a microscope equipped with a camera, sometimes referred to as high-efficiency photon detection system. Suitable photon detection systems include, but are not limited to, photodiodes and intensified CCD cameras. For example, as illustrated in FIG. 2, an intensified charge couple device (ICCD) camera can be used. The use of an ICCD camera to image individual fluorescent dye molecules in a fluid near a surface provides numerous advantages. For example, with an ICCD optical setup, it is possible to acquire a sequence of images (movies) of fluorophores.

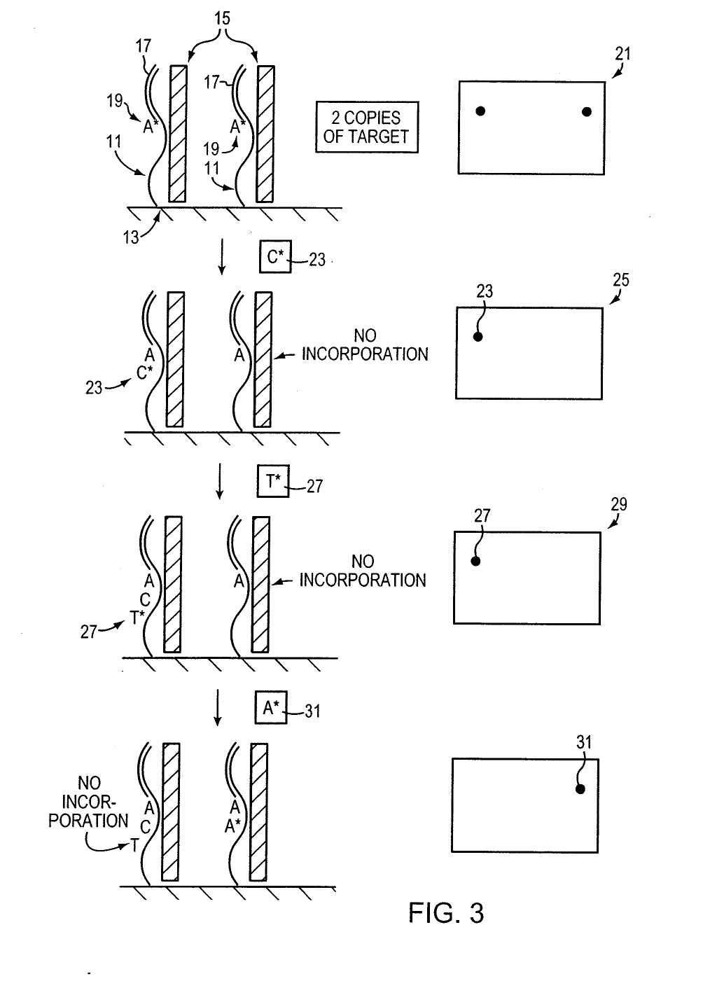

In this method, as shown in FIG. 3, a template single-stranded nucleic acid (target) 11 is attached to a solid substrate 13 and a single-stranded nucleic acid binding protein 15. A primer 17 also is bound to the template 11 and includes a labeled nucleotide 19. After an optional wash step, the locations of the two targets 11 are individually optically detectable as indicated by the substrate/surface 21. After photo-bleaching to render the primer label 19 undetectable, under conditions optimal for primer extension, a labeled nucleotide 23 and polymerase are added and extension is allowed to occur. If the labeled nucleotide 23 is incorporated, a detectable event occurs as indicated by the substrate/surface 25. If a plurality of nucleotides are used, a wash step may facilitate the reduction of any background resulting from the presence of any unincorporated nucleotide and/or other contaminants. Subsequent to photo-bleaching, primer extension is again allowed to occur with another labeled base 27 in the presence of a polymerase. Thereafter, the incorporation of labeled base 27 results in another detectable event as indicated in the substrate/surface 29. Another extension reaction with another labeled nucleotide 31 yields a detectable event as shown by the substrate/surface 33.

### Example 3

### FRET Labeling Methods

Nucleotide donor/acceptor. This method is generally similar to Example 2, however the nucleotides comprise either a donor and acceptor label. In this method, a primer is bound to a detectable label such as Cy3. The primer is selected to bind to the template nucleic acid that is attached to a surface. The surface is then washed and the positions of the Cy3-primed templates are recorded and bleached. Next, a Cy3 labeled nucleic acid and polymerase are introduced under optimal nucleic acid sequencing condition and the surface is washed. An image of the surface is then detected for incorporation of labeled nucleic acid. If there is no incorporation, the procedure is repeated with another nucleotide until a Cy3 labeled base incorporation onto the primer is detected. Once a Cy3 labeled nucleotide is detected, the label remains unbleached and the extension reaction is carried out in the presence of a Cy5 labeled nucleotide. After washing, an incorporation of a Cy5 labeled nucleotide results in an optically detectable event as the Cy5 label acts as an acceptor fluorophore from nearby Cy3 donor fluorophore. Subsequent to a Cy5 acceptor detection, the mixture is photobleached such that incorporation of another Cy5 labeled nucleotide is now detectable during subsequent extension reactions.

Single-stranded nucleic acid binding protein/Polymerase donor. A nucleic acid extension reaction is generally conducted as provided in Example 2, however either the single-stranded nucleic acid binding protein or polymerase comprises a donor fluorophore and the labeled nucleotides comprise an acceptor fluorophore. In this method, incorporation of a labeled nucleotide into the growing primer strand is visible during the detection phase of the reaction when a photon is transferred from either the donor single-stranded nucleic acid binding protein or the donor polymerase.

### Example 4

### Single-Stranded Nucleic Acid Binding Protein Anchoring and Stabilizing

In this example, a single-stranded nucleic acid binding protein is bound to a substrate. After binding a single-stranded nucleic acid binding protein to a substrate and washing away excess unbound single-stranded nucleic acid binding protein, a non-biotinylated single-stranded nucleic acid template is exposed and attached to the substrate/single-stranded nucleic acid binding protein complex. The complex is located on the substrate such that each template is individually optically resolvable.

Next, a labeled primer is introduced under conditions optimal for binding of the primer to the template. The substrate is then washed and incorporation of the labeled primer is detected. Optionally, the primer/template structure bound to the single-stranded nucleic acid binding protein may be photo-bleached to inactivate the detectable label from the primer, or if a FRET detection system is implemented, the label may be selected such that it includes a donor fluorophore.

Labeled nucleotides are then added to the reaction mixture along with a polymerase selected to catalyze the extension reaction. A reaction mixture can comprise only one labeled nucleotide or plurality of nucleotides. If a plurality of different nucleotides are included in the reaction mixture, each of the nucleotides can be differentially labeled. The labeled nucleotide(s) can be exposed to a polymerase and then the sequencing reaction can proceed as described herein.

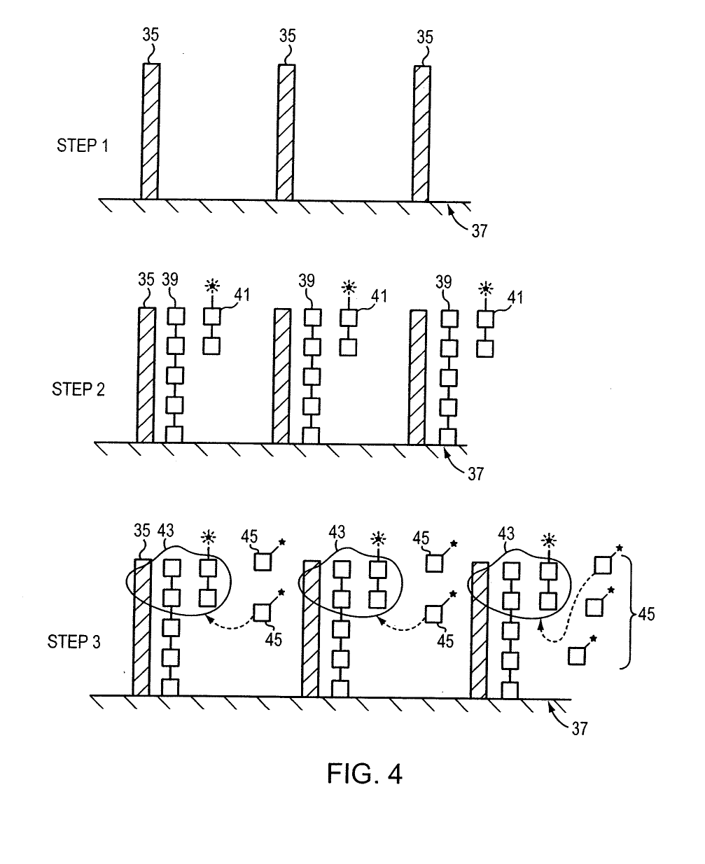

FIG. 4 illustrates an extension reaction using a single-stranded nucleic acid binding protein to fix a template onto a substrate. In step 1, a single-stranded nucleic acid binding protein 35 is attached to a substrate 37 and excess single-stranded nucleic acid binding protein 35 is washed away. In step 2, a target nucleic acid 39 is introduced and is bound by the single-stranded nucleic acid binding protein 35 to the substrate 37. After optional washing, a labeled primer 41 selected to hybridize to the target 39 is introduced and optically detected to confirm the presence of the target 39 and incorporation of the primer 41. In step 3, polymerase 43 is added to catalyze the primer extension in the presence of labeled nucleotides 45 under appropriate extension conditions. Primer extension is allowed to occur in a template dependent fashion with optional washes after each incorporation cycle and optional photo-bleaching based on the detection system utilized.

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are therefore intended to be embraced therein.

We claim:

1. A method for stabilizing a nucleic acid sequencing reaction, the method comprising the steps of:
exposing a mixture comprising a template, a polymerase, a primer, and at least one nucleotide to a single-stranded nucleic acid binding protein;
wherein said single-stranded nucleic acid binding protein binds to said template.

2. The method of claim 1, wherein said single-stranded nucleic acid binding protein increases the fidelity of said polymerase upon binding of said protein to said template.

3. The method of claim 1, wherein said nucleotide comprises a detectable label.

4. The method of claim 2, further comprising the detection of said nucleotide into said primer.

5. The method of claim 1, wherein said template is attached to a substrate such that it is individually optically resolvable.

6. The method of claim 1, wherein said nucleotide is labeled with a first label and wherein said single-stranded nucleic acid binding protein comprises a second label.

7. The method of claim 1, wherein said nucleotide is labeled with a first label and wherein said polymerase comprises a second label.

8. The method of claim 6 or 7, wherein said first and second labels are fluorophores.

9. The method of claim 8, wherein said detecting step comprises detecting coincident fluorescence emission of said labeled nucleotide and said second label.

10. The method of claim 1, wherein said nucleotide is a nucleotide analog.

11. The method of claim 10, wherein said nucleotide analog is a chain terminating analog.

12. A method for sequencing a polynucleotide, the method comprising the steps of:
(a) stabilizing a nucleic acid template/primer complex with a single-stranded nucleic acid binding protein;
(b) exposing said complex to a polymerase and at least one nucleotide capable of extending said primer;
(c) determining whether said nucleotide extends said primer;
(d) repeating said exposing and determining steps; and
(e) compiling a sequence of said polynucleotide based upon an order of nucleotides added to said primer.

13. The method of claim 12, wherein said template/primer complex is attached to a substrate such that it is individually optically resolvable.

14. The method of claim 12, further comprising the step of removing unincorporated nucleotide.

15. The method of claim 12, wherein said nucleotide comprises a detectable label.

16. The method of claim 15, further comprising the step of rendering said label undetectable subsequent to determining step.

17. The method of claim 15, wherein said nucleotide is labeled with a fluorophore.

18. The method of claim 15, wherein said determining step comprises optically detecting incorporation of said nucleotide.

19. The method of claim 12, wherein said nucleotide is labeled with a first label and wherein said single-stranded nucleic acid binding protein comprises a second label.

20. The method of claim 12, wherein said nucleotide is labeled with a first label and wherein said polymerase comprises a second label.

21. The method of claim 19 or 20, wherein said first and second labels are fluorophores.

22. The method of claim 21, wherein said detecting step comprises detecting coincident fluorescence emission of said labeled nucleotide and said second label.

23. The method of claim 12, wherein said nucleotide is a nucleotide analog.

24. The method of claim 23, wherein said nucleotide analog is a chain terminating analog.

25. A method for sequencing a nucleic acid template, the method comprising the steps of:
(a) exposing a nucleic acid template to a labeled nucleotide, a polymerase and a single-stranded nucleic acid binding protein under conditions that allow incorporation of said nucleotide into a primer attached to said template, wherein said single-stranded nucleic acid binding protein increases fidelity of said polymerase upon binding of said protein to said template;
(b) detecting incorporation of said nucleotide into said primer;
(c) repeating steps (a) and (b) at least once; and
(d) compiling a sequence of said template based upon an order of incorporated nucleotides.

26. The method of claim 25, wherein said template is attached to a substrate such that it is individually optically resolvable.

27. The method of claim 25, further comprising the step of removing unincorporated nucleotide.

28. The method of claim 25, further comprising the step of rendering said label undetectable subsequent to said detecting step.

29. The method of claim 25, wherein said nucleotide is labeled with a fluorophore.

30. The method of claim 29, wherein said detecting step comprises optically detecting incorporation of said nucleotide.

31. The method of claim 25, wherein said nucleotide is labeled with a first label and wherein said single-stranded nucleic acid binding protein comprises a second label.

32. The method of claim 25, wherein said nucleotide is labeled with a first label and wherein said polymerase comprises a second label.

33. The method of claim 31 or 32, wherein said first and second labels are fluorophores.

34. The method of claim 33, wherein said detecting step comprises detecting coincident fluorescence emission of said labeled nucleotide and said second label.

35. The method of claim 25, wherein said nucleotide is a nucleotide analog.

36. The method of claim 35, wherein said nucleotide analog is a chain terminating analog.

37. A method for sequencing a nucleic acid template, the method comprising the steps of:
(a) exposing a nucleic acid template to a nucleotide, a polymerase and a single-stranded nucleic acid binding protein under conditions that allow incorporation of said nucleotide into a primer attached to said template, said nucleotide comprising a first label, and said single-stranded nucleic acid binding protein comprising a second label,
wherein one of said first and second labels comprises a donor fluorophore and the other of said labels comprises an acceptor fluorophore; and
wherein, upon binding of said single-stranded nucleic acid binding protein to said template and incorporation of said nucleotide analogue into said primer, said acceptor fluorophore is optically detectable;
(b) detecting said acceptor fluorophore, thereby to detect incorporation of said nucleotide into said primer;
(c) repeating steps (a) and (b) at least once; and
(d) compiling a sequence of said template based upon an order of incorporated nucleotides.

38. The method of claim 37, wherein said template is attached to a substrate such that it is individually optically resolvable.

39. The method of claim 25 or 37, wherein said single-stranded nucleic acid binding protein is attached to a substrate, and wherein said single-stranded nucleic acid binding protein binds said template, thereby attaching said template to said substrate.

40. The method of claim 37, further comprising the step of removing unincorporated nucleotide.

41. The method of claim 37, further comprising the step of rendering said acceptor fluorophore undetectable subsequent to said detecting step.

42. A method for sequencing a nucleic acid template, the method comprising the steps of:
(a) exposing a nucleic acid template to a labeled nucleotide and a polymerase under conditions that allow incorporation of said nucleotide into a primer attached to said template, said template being attached to a substrate-bound single-stranded nucleic acid binding protein such that said template is individually optically resolvable;
(b) detecting incorporation of said nucleotide into said primer;
(c) repeating steps (a) and (b) at least once; and
(d) compiling a sequence of said template based upon an order of incorporated nucleotides.

43. The method of claim 42, wherein said single-stranded nucleic acid binding protein increases fidelity of said polymerase upon binding of said protein to said template.

44. The method of claim 42, wherein said nucleotide is labeled with a first label and wherein said single-stranded nucleic acid binding protein comprises a second label.

45. The method of claim 44, wherein said first and second labels are fluorophores.
